(19) United States
(12) Patent Application Publication
(10) Pub. No.: US 2024/0120022 A1
(43) Pub. Date: Apr. 11, 2024

(54) PREDICTING PROTEIN AMINO ACID SEQUENCES USING GENERATIVE MODELS CONDITIONED ON PROTEIN STRUCTURE EMBEDDINGS

(71) Applicant: DeepMind Technologies Limited, London (GB)

(72) Inventors: Andrew W. Senior, London (GB); Simon Kohl, London (GB); Jason Yim, London (GB); Russell James Bates, London (GB); Catalin-Dumitru Ionescu, London (GB); Charlie Thomas Curtis Nash, London (GB); Ali Razavi-Nematollahi, London (GB); Alexander Pritzel, London (GB); John Jumper, London (GB)

(21) Appl. No.: 18/275,933

(22) PCT Filed: Jan. 27, 2022
(86) PCT No.: PCT/EP2022/051942

§ 371 Date: Aug. 4, 2023

Related U.S. Application Data
(60) Provisional application No. 63/146,091, filed on Feb. 5, 2021.

(51) Int. Cl. G16B 15/00; G16B 30/20; G16B 40/20

(52) U.S. Cl. CPC G16B 15/00; G16B 30/20; G16B 40/20

(57) ABSTRACT

Methods, systems, and apparatus, including computer programs encoded on a computer storage medium, for performing protein design. In one aspect, a method comprises: processing an input characterizing a target protein structure of a target protein using an embedding neural network having a plurality of embedding neural network parameters to generate an embedding of the target protein structure of the target protein; determining a predicted amino acid sequence of the target protein based on the embedding of the target protein structure, comprising: conditioning a generative neural network having a plurality of generative neural network parameters on the embedding of the target protein structure; and generating, by the generative neural network conditioned on the embedding of the target protein structure, a representation of the predicted amino acid sequence of the target protein.

### BACKGROUND

This specification relates to designing proteins to achieve a specified protein structure.

A protein is specified by one or more sequences of amino acids. An amino acid is an organic compound which includes an amino functional group and a carboxyl functional group, as well as a side-chain (i.e., group of atoms) that is specific to the amino acid.

Protein folding refers to a physical process by which a sequence of amino acids folds into a three-dimensional configuration. The structure of a protein defines the three-dimensional configuration of the atoms in the amino acid sequence of the protein after the protein undergoes protein folding. When in a sequence linked by peptide bonds, the amino acids may be referred to as amino acid residues.

Predictions can be made using machine learning models. Machine learning models receive an input and generate an output, e.g., a predicted output, based on the received input. Some machine learning models are parametric models and generate the output based on the received input and on values of the parameters of the model. Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes a protein design system implemented as computer programs on one or more computers in one or more locations that processes data defining a protein structure to generate an amino acid sequence of a protein that is predicted to fold into the protein structure.

As used throughout this specification, the term “protein” may be understood to refer to any biological molecule that is specified by one or more sequences of amino acids. For example, the term protein may be understood to refer to a protein domain (i.e., a portion of an amino acid sequence that can undergo protein folding nearly independently of the rest of the amino acid sequence) or a protein complex (i.e., that is specified by multiple associated amino acid sequences).

Throughout this specification, an embedding refers to an ordered collection of numerical values, e.g., a vector or matrix of numerical values.

According to a first aspect, there is provided a method performed by one or more data processing apparatus, the method comprising: processing an input characterizing a target protein structure of a target protein using an embedding neural network having a plurality of embedding neural network parameters to generate an embedding of the target protein structure of the target protein; determining a predicted amino acid sequence of the target protein based on the embedding of the target protein structure, comprising: conditioning a generative neural network having a plurality of generative neural network parameters on the embedding of the target protein structure; and generating, by the generative neural network conditioned on the embedding of the target protein structure, a representation of the predicted amino acid sequence of the target protein; processing the representation of the predicted amino acid sequence using a protein folding neural network to generate a representation of a predicted protein structure of a protein having the predicted amino acid sequence; determining a structural similarity measure between: (i) the predicted protein structure of the protein having the predicted amino acid sequence, and (ii) the target protein structure; determining gradients of the structural similarity measure with respect to the embedding neural network parameters and the generative neural network parameters; and adjusting current values of the embedding neural network parameters and the generative neural network parameters using the gradients of the structural similarity measure.

In some implementations, determining gradients of the structural similarity measure with respect to the embedding neural network parameters and the generative neural network parameters comprises: backpropagating gradients of the structural similarity measure through the protein folding neural network into the generative neural network and the embedding neural network.

In some implementations, the method further comprises: processing the representation of the predicted protein structure of the protein having the predicted amino acid sequence using a discriminator neural network to generate a realism score that defines a likelihood that the predicted amino acid sequence was generated using the generative neural network; determining gradients of the realism score with respect to the embedding neural network parameters and the generative neural network parameters; and adjusting current values of the embedding neural network parameters and the generative neural network parameters using the gradients of the realism score.

In some implementations, determining gradients of the realism score with respect to the embedding neural network parameters and the generative neural network parameters comprises: backpropagating gradients of the realism score through the discriminator neural network and the protein folding neural network into the generative neural network and the embedding neural network.

In some implementations, generating the realism score comprises processing an input that includes both: (i) the representation of the predicted protein structure having the predicted amino acid sequence, and (ii) the representation of the predicted amino acid sequence, using the discriminator neural network.

In some implementations, the method further comprises: determining a sequence similarity measure between: (i) the predicted amino acid sequence of the target protein, and (ii) a target amino acid sequence of the target protein; determining gradients of the sequence similarity measure with respect to the embedding neural network parameters and the generative neural network parameters; and adjusting current values of the embedding neural network parameters and the generative neural network parameters using the gradients of the sequence similarity measure.

In some implementations, the embedding neural network input characterizing the target protein structure comprises: (i) a respective initial pair embedding corresponding to each pair of amino acids in the target protein that characterizes a distance between the pair of amino acids in the target protein structure, and (ii) a respective initial single embedding corresponding to each amino acid in the target protein.

In some implementations, the embedding neural network comprises a sequence of update blocks, wherein each update block has a respective set of update block parameters and performs operations comprising: receiving current pair embeddings and current single embeddings; updating the current single embeddings, in accordance with values of the update block parameters of the update block, based on the current pair embeddings; and updating the current pair embeddings, in accordance with the values of the update block parameters of the update block, based on the updated single embeddings; wherein a first update block in the sequence of update blocks receives the initial pair embeddings and the initial single embeddings; and wherein a final update block in the sequence of update blocks generates final pair embeddings and final single embeddings.

In some implementations, generating the embedding of the target protein structure of the target protein comprises: generating the embedding of the target protein structure of the target protein based on the final pair embeddings, the final single embeddings, or both.

In some implementations, updating the current single embeddings based on the current pair embeddings comprises: updating the current single embeddings using attention over the current single embeddings, wherein the attention is conditioned on the current pair embeddings.

In some implementations, updating the current single embeddings using attention over the current single embeddings comprises: generating, based on the current single embeddings, a plurality of attention weights; generating, based on the current pair embeddings, a respective attention bias corresponding to each of the attention weights; generating a plurality of biased attention weights based on the attention weights and the attention biases; and updating the current single embeddings using attention of the current single embeddings based on the biased attention weights.

In some implementations, updating the current pair embeddings based on the updated single embeddings comprises: applying a transformation operation to the updated single embeddings; and updating the current pair embeddings by adding a result of the transformation operation to the current pair embeddings.

In some implementations, the transformation operation comprises an outer product operation.

In some implementations, updating the current pair embeddings based on the updated single embeddings further comprises, after adding the result of the transformation operation to the current pair embeddings: updating the current pair embeddings using attention over the current pair embeddings, wherein the attention is conditioned on the current pair embeddings.

In some implementations, generating, by the generative neural network conditioned on the embedding of the target protein structure, the representation of the predicted amino acid sequence of the target protein comprises: processing the embedding of the target protein structure to generate data defining parameters of a probability distribution over a latent space; sampling a latent variable from the latent space in accordance with the probability distribution over the latent space; and processing the latent variable sampled from the latent space to generate the representation of the predicted amino acid sequence.

In some implementations, generating, by the generative neural network conditioned on the embedding of the target protein structure, the representation of the predicted amino acid sequence of the target protein comprises, for each position in the predicted amino acid sequence: processing: (i) the embedding of the target protein structure, and (ii) data defining amino acids at any preceding positions in the predicted amino acid sequence, to generate a probability distribution over a set of possible amino acids; and sampling an amino acid for the position in the predicted amino acid sequence from the set of possible amino acids in accordance with the probability distribution over the set of possible amino acids.

In some implementations, the method further comprises obtaining a representation of a three-dimensional shape and size of a surface portion of a target body, and obtaining the target protein structure as a structure including a portion which has a shape and size complementary to the shape and size of the surface portion of the target body.

According to another aspect, there is provided a method of obtaining a ligand to a binding target, the method comprising: obtaining a representation of a three-dimensional shape and size of a surface portion of the binding target for the ligand; obtaining a target protein structure as a structure including a portion which has a shape and size complementary to the shape and size of the surface portion of the binding target; determining an amino acid sequence of one or more corresponding target proteins predicted to have the target protein structure using an embedding neural network and a generative neural network; evaluating an interaction of the one or more target proteins with the binding target; and selecting one or more of the target proteins as the ligand dependent on a result of the evaluating.

In some implementations, the binding target comprises a receptor or enzyme, and wherein the ligand is an agonist or antagonist of the receptor or enzyme.

In some implementations, the binding target is an antigen which comprises a virus protein or a cancer cell protein.

In some implementations, the binding target is a protein associated with a disease, and the target protein is selected as a diagnostic antibody marker of the disease.

In some implementations, the generating, by the generative neural network conditioned on the embedding of the target protein structure, a representation of the predicted amino acid sequence of the target protein, is conditioned on an amino acid sequence which is to be included in the predicted amino acid sequence.

According to another aspect there is provided a method comprising: determining an amino acid sequence of a target protein predicted to have a target protein structure using an embedding neural network and a generative neural network; and physically synthesizing the target protein having the determined amino acid sequence.

According to another aspect there is provided a method performed by one or more data processing apparatus, the method comprising: processing an input characterizing a target protein structure of a target protein using an embedding neural network having a plurality of embedding neural network parameters to generate an embedding of the target protein structure of the target protein; determining a predicted amino acid sequence of the target protein based on the embedding of the target protein structure, comprising: conditioning a generative neural network having a plurality of generative neural network parameters on the embedding of the target protein structure; and generating, by the generative neural network conditioned on the embedding of the target protein structure, a representation of the predicted amino acid sequence of the target protein; wherein the embedding neural network and the generative neural network have been jointly trained by operations comprising, for each training protein in a set of training proteins: generating a predicted amino acid sequence of the training protein using the embedding neural network and the generative neural network; processing the representation of the predicted amino acid sequence of the training protein using a protein folding neural network to generate a representation of a predicted protein structure of a protein having the predicted amino acid sequence; determining a structural similarity measure between: (i) the predicted protein structure of the protein having the predicted amino acid sequence, and (ii) a training protein structure of the training protein; determining gradients of the structural similarity measure with respect to the embedding neural network parameters and the generative neural network parameters; and adjusting values of the embedding neural network parameters and the generative neural network parameters using the gradients of the structural similarity measure.

According to another aspect there is provided a system comprising: one or more computers; and one or more storage devices communicatively coupled to the one or more computers, wherein the one or more storage devices store instructions that, when executed by the one or more computers, cause the one or more computers to perform the operations of the methods described herein.

One or more non-transitory computer storage media storing instructions that when executed by one or more computers cause the one or more computers to perform the operations of the methods described herein.

Particular embodiments of the subject matter described in this specification can be implemented so as to realize one or more of the following advantages.

The protein design system described in this specification can predict the amino acid sequence of a protein based on the structure of the protein. More specifically, the protein design system processes a set of structure parameters defining a protein structure to generate a protein structure embedding, and generates an amino acid sequence of a protein that is predicted to have the protein structure using a generative neural network that is conditioned on the protein structure embedding.

To generate the protein structure embedding, the protein design system can initialize a respective “pair” embedding corresponding to each pair of amino acids in the protein, and a respective “single” embedding corresponding to each amino acid in the protein. The protein design system uses an embedding neural network to alternate between updating the pair embeddings using the single embeddings and updating the single embeddings using the pair embeddings. Updating the pair embeddings using the single embeddings enriches the information content of the pair embeddings using the complementary information encoded in the single embeddings. Conversely, updating the single embeddings using the pair embeddings enriches the information content of the single embeddings using the complementary information encoded in the pair embeddings. After updating the pair embeddings and the single embeddings, the protein design system generates the protein structure embedding based on the pair embeddings, the single embeddings, or both. The enriched information content of the pair embeddings and the single embeddings causes the protein structure embedding to encode information that is more relevant to predicting an amino acid sequence that folds into the protein structure, and thereby enables the protein design system to predict the amino acid sequence more accurately.

The training system described in this specification can train the protein design system to optimize a “structure loss.” To evaluate the structure loss, the training system can process a “target” protein structure using the protein design system to generate a corresponding amino acid sequence, and then process the amino acid sequence using a protein folding neural network to predict the structure of a protein having the amino acid sequence. The training system determines the structure loss based on an error between: (i) the predicted protein structure of the protein generated by the protein design system, and (ii) the target protein structure. The structure loss evaluates the accuracy of the protein design system in “structure space,” i.e., in the space of possible protein structures. In contrast, a “sequence loss” that measures a similarity between: (i) a the amino acid sequence of a training example, and (ii) the amino acid sequence generated by the protein design system upon receiving as input the protein structure of the training example, evaluates the accuracy of the protein design system in “sequence space,” i.e., in the space of possible amino acid sequences. Therefore, updates to the protein design system parameters generated using the structure loss are complementary to those generated using the sequence loss. Training the protein design system to optimize the structure loss can enable the protein design system to achieve an acceptable performance (e.g., prediction accuracy, such as a high success rate in generating amino acid sequences corresponding to proteins which do indeed have the target protein structure, to within a certain level of tolerance) over fewer training iterations (thereby reducing consumption of computational resources, e.g., memory and computing power, during training), and can increase prediction accuracy of the trained protein design system.

The training system can also train the protein design system to optimize a “realism loss” that characterizes whether proteins generated by protein design system have the characteristics of “real” proteins, e.g., that can exist in the natural world. For example, the realism loss can implicitly characterize whether a protein generated by the protein design system would violate bio-chemical constraints that apply to real proteins. Training the protein design system to optimize the realism loss can enable the protein design system to achieve an acceptable performance (e.g., prediction accuracy) over fewer training iterations (thereby reducing consumption of computational resources, e.g., memory and computing power, during training), and can increase prediction accuracy of the trained protein design system. Moreover, the training system evaluates the realism loss using a discriminator neural network that can automatically learn to identify complex, high-level features that distinguish “synthetic proteins” generated by the protein design system from real proteins, thereby obviating any requirement to manually design functions that evaluate protein realism.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example protein design system.

FIG. 2 shows an example architecture of an embedding neural network that is included in the protein design system.

FIG. 3 shows an example architecture of an update block of the embedding neural network.

FIG. 4 shows an example architecture of a single embedding update block.

FIG. 5 shows an example architecture of a pair embedding update block.

FIG. 6 shows an example training system for training a protein design system.

FIG. 7 is a flow diagram of an example process for determining a predicted amino acid sequence of a target protein having a target protein structure.

Like reference numbers and designations in the various drawings indicate like elements.

### DETAILED DESCRIPTION

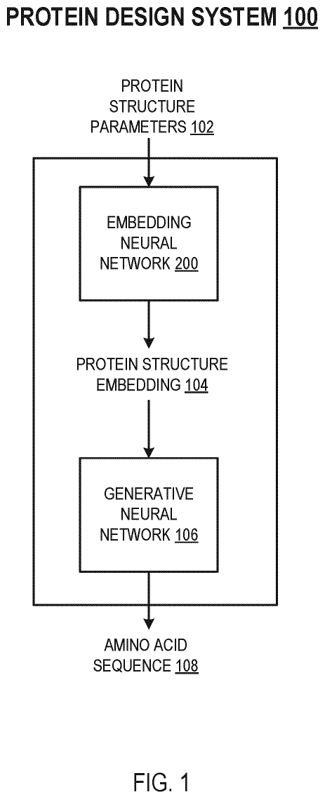

FIG. 1 shows an example protein design system 100. The protein design system 100 is an example of a system implemented as computer programs on one or more computers in one or more locations in which the systems, components, and techniques described below are implemented.

The protein design system 100 is configured to process a set of structure parameters 102 representing a protein structure to generate a representation of an amino acid sequence 108 of a protein that is predicted to achieve the protein structure, i.e., after undergoing protein folding.

The protein design system 100 can receive the structure parameters 102 representing the protein structure, e.g., from a remotely located user of the protein design system 100 through an application programming interface (API) made available by the protein design system 100.

The protein structure parameters 102 defining the protein structure can be represented in a variety of formats. A few examples of possible formats of the protein structure parameters 102 are described in more detail next.

In some implementations, the protein structure parameters 102 are expressed as a distance map. The distance map defines, for each pair of amino acids in the protein, the respective distance between the pair of amino acids in the protein structure. The distance between a first amino acid and a second amino acid in a protein structure can refer to a distance between a specified atom in the first amino acid and a specified atom in the second amino acid in the protein structure. The specified atom may be, e.g., the alpha carbon atom, i.e., the carbon atom in the amino acid to which the amino functional group, the carboxyl functional group, and the side-chain of the amino acid are bonded. The distance between amino acids can be measured, e.g., in Angstroms.

In some implementations, the structure parameters are expressed as a sequence of three-dimensional (3D) numerical coordinates (e.g., represented as 3D vectors), where each coordinate represents the position (in some given frame of reference) of a corresponding atom in an amino acid of the protein. For example, the structure parameters may be a sequence of 3D numerical coordinates representing the respective positions of the alpha carbon atoms in the amino acids of the protein. As a further example, the structural parameters can define backbone atom torsion angles of the amino acids in the protein.

The amino acid sequence 108 generated by the protein design system 100 defines which amino acid, from a set of possible amino acids, occupies each position in the amino acid sequence of the protein. The set of possible amino acids can include 20 amino acids, e.g., alanine, arginine, asparagine, etc.

The protein design system 100 generates the amino acid sequence 108 of the protein that is predicted to achieve the protein structure using: (i) an embedding neural network 200, and (ii) a generative neural network 106, which are each described in more detail next.

The embedding neural network 200 is configured to process the protein structure parameters 102 to generate an embedding of the protein structure, referred to as the protein structure embedding 104. The protein structure embedding 104 implicitly represents various features of the protein structure that are relevant to predicting the amino acid sequence of the protein that achieves the protein structure.

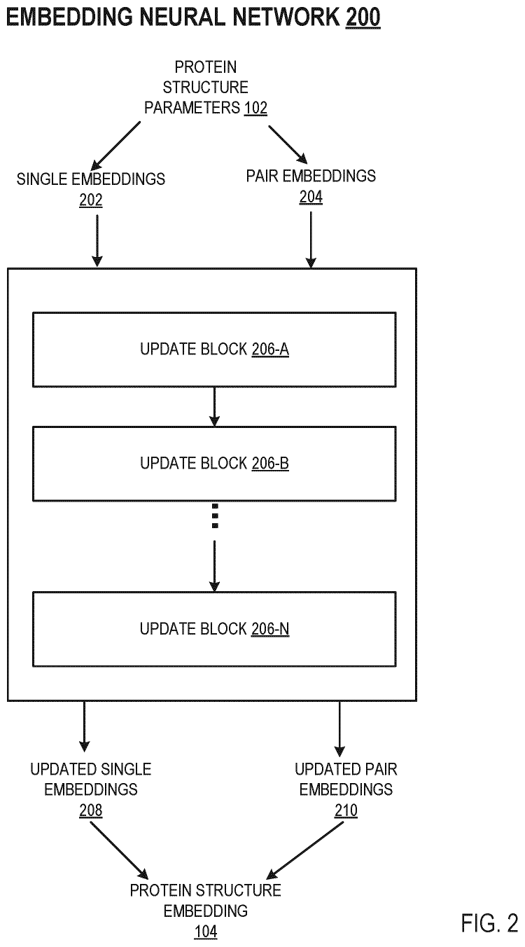

The embedding neural network 200 can have any appropriate neural network architecture that enables it to perform its described functions, e.g., processing protein structure parameters 102 defining a protein structure to generate a protein structure embedding 104. An example architecture of the embedding neural network 200 is described in more detail with reference to FIG. 2.

The generative neural network 106 is configured to process the protein structure embedding 104 to generate data defining the amino acid sequence 108 of a protein that is predicted to achieve the protein structure. Providing the protein structure embedding 104 to the generative neural network 106, to be processed by the generative neural network 106 as part of generating the amino acid sequence 108, can be referred to as “conditioning” the generative neural network 106 on the protein structure embedding 104.

The generative neural network 106 can have any appropriate generative neural network architecture that enables it to perform its described function, i.e., generating an amino acid sequence of a protein that is predicted to achieve the protein structure. In particular, the generative neural network can include any appropriate neural network layers, e.g., convolutional layers, fully-connected layers, self-attention layers, etc., connected in any appropriate configuration (e.g., as a linear sequence of layers). A few examples of the neural network operations that can be performed by the generative neural network 106 to generate the amino acid sequence 108 are described in more detail next.

In some implementations, the generative neural network 106 is configured to process the protein structure embedding 104 using one or more neural network layers, e.g., fully-connected neural network layers, to generate data defining the parameters of a probability distribution over a latent space. The latent space can be, e.g., an N-dimensional Euclidean space, i.e., I′ZN and the parameters defining the probability distribution can be a mean vector and a covariance matrix of a Normal probability distribution over the latent space. The generative neural network 106 can then sample a latent variable from the latent space in accordance with the probability distribution over the latent space. The generative neural network 106 can process the sampled latent variable (and, optionally, the protein structure embedding 104) using one or more neural network layers (e.g., fully-connected neural network layers) to generate, for each position in the amino acid sequence 108, a respective probability distribution over the set of possible amino acids. The generative neural network 106 can then sample a respective amino acid for each position in the amino acid sequence, i.e., in accordance with the corresponding probability distribution over the set of possible amino acids, and output the resulting amino acid sequence 108.

Alternatively to or in combination with sampling a single, “global” latent variable (as described above), the generative neural network 106 can be configured to sample multiple “local” latent variables. In one example, the embedding neural network 200 may generate a protein structure embedding 104 that includes a respective “single” embedding corresponding to each position in the amino acid sequence of the protein (as will be described in more detail with reference to FIG. 2). In this example, the generative neural network 106 can, for each position in the amino acid sequence of the protein, process the single embedding for the position using one or more neural network layers to generate a corresponding probability distribution over a latent space. The generative neural network 106 can then sample a local latent variable corresponding to the position in the amino acid sequence from the latent space in accordance with the probability distribution over the latent space. The generative neural network 106 can subsequently process the local latent variables as part of generating the output amino acid sequence 108.

In some implementations, the generative neural network 106 is an autoregressive neural network that, starting from the first position in the amino acid sequence, sequentially selects the amino acid at each position in the amino acid sequence. To select the amino acid at a current position in the amino acid sequence 108, the generative neural network processes: (i) the protein structure embedding 104, and (ii) data defining the amino acids at any preceding positions in the amino acid sequence 108, using one or more neural network layers to generate a probability distribution over the set of possible amino acids for the current position in the amino acid sequence. The generative neural network does not process data defining the amino acids at positions subsequent to the current position in the amino acid sequence because these amino acids have not yet been selected, i.e., at the time that the amino acid at the current position is being selected. The data defining the amino acids at the preceding positions in the amino acid sequence may include, e.g., a respective one-hot vector corresponding to each preceding position that defines the identity of the amino acid at the preceding position. After generating the probability distribution over the set of possible amino acids for the current position in the amino acid sequence, the generative neural network can then select the amino acid at the current position by sampling from the set of possible amino acids in accordance with the probability distribution.

Optionally, rather than generating a single amino acid sequence 108, the protein design system 100 can use the generative neural network 106 to generate a set of multiple amino acid sequences 108 that are each predicted to fold into the protein structure. For example, if the generative neural network 106 autoregressively samples the amino acid at each position in the amino acid sequence, as described above, then the generative neural network can repeat the autoregressive sampling process multiple times to generate multiple amino acid sequences. As another example, if the generative neural network 106 generates the amino acid sequence processing a latent variable that is sampled from a latent space (as described above), then the generative neural network can sample multiple latent variables and process each sampled latent variable to generate a respective amino acid sequence.

Amino acid sequences 108 generated by the protein design system 100 can be used in any of a variety of ways. For example, a protein having the amino acid sequence 108 can be physically synthesized. Experiments can be performed to determine whether the protein folds into the desired protein structure.

One application of the protein design system 100 is to produce elements having a desired three-dimensional shape and size specified by the target protein structure. In effect, this provides a 3D printer on a microscopic scale. The elements may have dimensions of 10s of microns, or even less. For example, the largest dimension of the physically synthesized protein (i.e. the length of the protein along the axis for which that length is highest) may be under 50 microns, under 5 microns or even under 1 micron. The present disclosure thus provides a novel technique for fabrication of micro-components having a desired three-dimensional shape and size.

For example, the target protein structure may specify that the target protein is elongate, i.e. the protein has extents in two transverse dimensions which are much smaller (e.g., at least 5 times smaller) than the extent of the protein in a third dimension transverse to the first two dimensions. This allows the target protein, once synthesized, to pass through a membrane containing apertures which are only slightly wider than the extent of the target protein in the two transverse dimensions.

In another example, the target protein structure may specify that the target protein is laminar, so that the synthesized target protein has the form of a platelet.

In a further example, the synthesized target protein could provide a component of a (microscopic) mechanical system having a desired shape and size defined by the target protein structure, for example a wheel, a rack, a pinion, or a lever.

In a further example, the target protein structure could be chosen to define a structure including a chamber for receiving at least part of another body (such as a chemically-active body such as a measure of a drug compound, a magnetic body or a radioactive body). The other body may be contained within the chamber. For example, it may be present when the target protein is synthesized, so that as the target protein folds to form the target protein structure, the other body becomes trapped within the chamber. There it may be prevented from interacting with nearby molecules, e.g., until a chemical reaction occurs to break down the protein structure and release the additional body. In some cases, only a part of the other body may be inserted into the chamber, so that the protein acts as a cap which covers that part of the other body, e.g., until a chemical reaction occurs transforming the protein to release the other body.

Furthermore, the shape and size of the protein can be selected to allow it to be placed in close contact to a surface of another body, a “binding target”, such as another microscopic body. For example, the binding target could have a surface of which a portion has a known three-dimensional shape and size. Using the known three-dimensional shape and size, a complementary shape can be defined, having a defined size. The target protein structure may be calculated based on the complementary shape, e.g., such that one side of the target protein has the complementary shape. Thus, the protein design system 100 can be used to obtain a protein which, once fabricated, includes the complementary shape of the defined size (e.g., on one side of the protein), and fits against the portion of the surface of the binding target, like a key fitting into a lock. The synthesized target molecule may in some cases be retained against the binding target, e.g., by attractive forces between the respective portions of the target protein and the binding target which come into close contact. The term “complementary” means that the target protein may be placed against the binding target with the volume between them being below a certain threshold. Furthermore, the complementary shape may be chosen such that, when the target protein is against the binding target, a plurality of specified points on the target protein are within a certain distance of corresponding points (e.g., binding sites) on the binding target.

Optionally, the protein design system 100 may be used more than once, to generate amino acid sequences for a plurality of corresponding target proteins which the protein design system predicts will have the target protein structure. The interaction of the plurality of target proteins with the binding target may be evaluated (e.g., computationally, or by synthesizing the target proteins and then measuring the interaction experimentally). Based on the evaluation results, one of the plurality of target proteins may be selected.

The target protein (or the selected one of the plurality of target proteins) may thus act as a ligand which binds to the binding target. If the binding target is also a protein molecule, it may be regarded as a receptor, and the target protein may act as a ligand to that receptor. The ligand may be a drug or act as a ligand to an industrial enzyme. The ligand may be an agonist or antagonist of the receptor or enzyme. Furthermore, the binding target may be an antigen which comprises a virus protein or a cancer cell protein. If the binding target is a biomolecule, the ligand may be such as to have a therapeutic effect. The protein may, for example, have the effect of inhibiting the binding target from participating in interactions with other molecules (e.g., chemical reactions), i.e. by preventing those molecules from coming into contact with the surface of the binding target. In one case, the binding target might be a cell (e.g., a human cell) or a component of a cell, and the protein might bind to the cell surface to protect the cell from interacting with harmful molecules. In a further case, the binding target might be harmful, e.g., a virus or a cancer cell, and by binding to it, the protein might prevent the binding target from taking part in a certain process, e.g., a reproductive process or an interaction with a cell of a host.

Alternatively, if the binding target is a protein associated with a disease, the target protein may be used as a diagnostic antibody marker of the disease.

In some cases, it may be desirable for the protein to have desired amino acids at certain locations of the structure, e.g., at exposed locations of the structure where they can be involved in chemical interactions with other molecules. In this case, it may be desirable to modify the amino acid sequence 108 to incorporate the desired amino acids. In this case, a test may be carried out (e.g., using a protein folding neural network, or a real-world experiment) to determine the structure of the protein having the amino modified acid sequence, to verify that it retains the target protein structure.

Alternatively, the operation of the generative neural network 106 may be modified to increase the likelihood of the desired amino acids being included in the generated amino acid sequence at the desired locations. For example, in the case that the generator neural network 106 samples the amino acid probability distribution at each position in the amino acid sequence, as described above, the sampling may be biased to increase the likelihood of the desired amino acids being includes in the generated amino acid sequence at the desired positions.

A further application of the protein design system 100 is in the field of peptidomimetics, in which proteins, or protein-like, chains are designed to mimic a peptide. Using the present method, a protein may be generated which has a shape and size which mimic the shape and size of the pre-existing peptide.

FIG. 2 shows an example architecture of an embedding neural network 200 that is included in a protein design system, e.g., the protein design system 100 that is described with reference to FIG. 1. The embedding neural network 200 is configured to generate a protein structure embedding 104 that represents a protein structure defined by a set of protein structure parameters 102.

To generate the protein structure embedding 104, the protein design system initializes: (i) a respective “single” embedding corresponding to each amino acid in the amino acid sequence of the protein, and (ii) a respective “pair” embedding corresponding to each pair of amino acids in the amino acid sequence of the protein.

The protein design system initializes the single embeddings 202 using “positional encoding,” i.e., such that the single embedding corresponding to each amino acid in the amino acid sequence is initialized as a function of the index of the position of the amino acid in the amino acid sequence. For example, the protein design system can initialize the single embeddings using the sinusoidal positional encoding technique described with reference to A. Vaswani et al., “Attention is all you need,” 21st Conference on Neural Informational Processing Systems (NIPS 2017).

The protein design system initializes the pair embedding corresponding to each pair of amino acids in the amino acid sequence based on the distance between the pair of amino acids in the protein structure, i.e., as defined by the protein structure parameters 102. More specifically, each entry in the pair embedding for a pair of amino acids is associated with a respective distance interval, e.g., [0, 2) Angstroms, [2,4) Angstroms, etc. The distance between the pair of amino acids will be included in one of these distance intervals, and the protein design system sets the value of the corresponding entry in the pair embedding to 1 (or some other predetermined value). The protein design system sets the values of the remaining entries in the embedding to 0 (or some other predetermined value).

The embedding neural network 200 processes the single embeddings 202 and the pair embeddings 204 using a sequence of update blocks 206-A-N to generate updated single embeddings 208 and updated pair embeddings 210. Throughout this specification, a “block” refers to a portion of a neural network, e.g., a subnetwork of the neural network that includes one or more neural network layers.

Each update block in the embedding neural network 200 is configured to receive a block input that includes a set of single embeddings and a set of pair embeddings, and to process the block input to generate a block output that includes updated single embeddings and updated pair embeddings.

The protein design system provides the single embeddings 202 and the pair embeddings 204 to the first update block (i.e., in the sequence of update blocks). The first update block processes the single embeddings 202 and the pair embeddings 204 to generate updated single embeddings and updated pair embeddings.

For each update block after the first update block, the embedding neural network 200 provides the update block with the single embeddings and the pair embeddings generated by the preceding update block, and provides the updated single embeddings and the updated pair embeddings generated by the update block to the next update block.

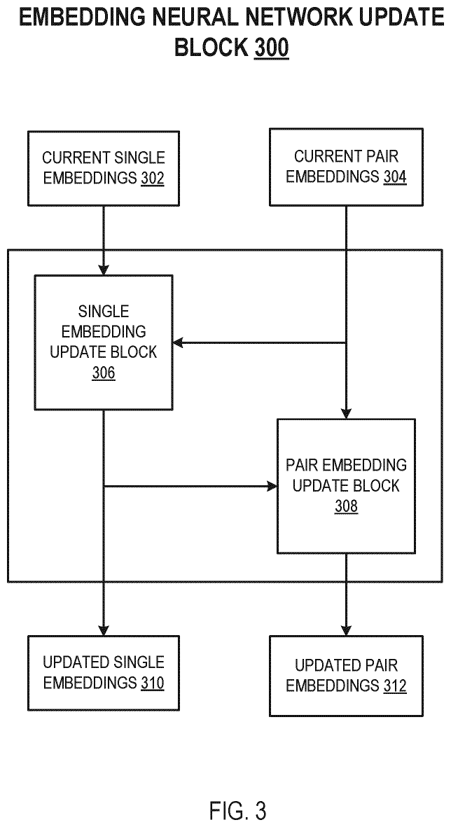

The embedding neural network 200 gradually enriches the information content of the single embeddings 202 and the pair embeddings 204 by repeatedly updating them using the sequence of update blocks 206-A-N, as will be described in more detail with reference to FIG. 3.

The protein design system generates the protein structure embedding 104 using the updated single embeddings 208, the updated pair embeddings 210, or both, that are generated by the final update block of the embedding neural network 200. For example, the protein design system can identify the protein structure embedding 104 as the updated single embeddings 208 alone, the updated pair embeddings 210 alone, or the concatenation of the updated single embeddings 208 and the updated pair embeddings 210.

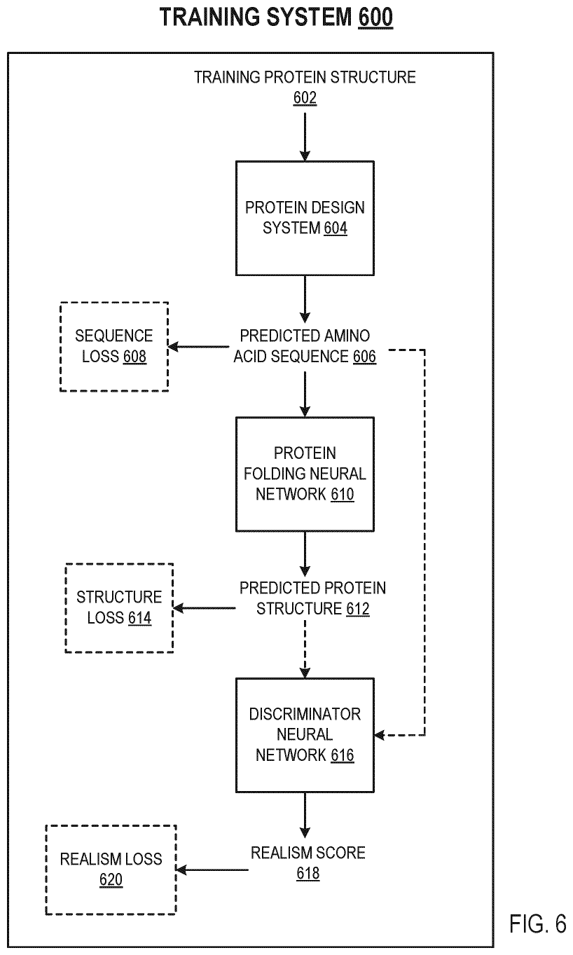

During training of the protein design system, which will be described in more detail with reference to FIG. 6, the embedding neural network 200 can include one or more neural network layers that process the updated single embeddings 208 to predict the amino acid sequence of the protein. The accuracy of the predicted amino acid sequence is evaluated using a loss function, e.g., a cross-entropy loss function, and gradients of the loss function are backpropagated through the embedding neural network to encourage the single embeddings to encode information that is relevant to predicting the amino acid sequence.

The embedding neural network 200 can also include one or more neural network layers that process the updated pair embeddings 210 to predict a distance map that defines the respective distance between each pair of amino acids in the protein structure. The accuracy of the predicted distance map is evaluated using a loss function, e.g., a cross-entropy loss function, and gradients of the loss function are backpropagated through the embedding neural network to encourage the pair embeddings to encode information characterizing the protein structure.

FIG. 3 shows an example architecture of an update block 300 of the embedding neural network 200, i.e., as described with reference to FIG. 2.

The update block 300 receives a block input that includes the current single embeddings 302 and the current pair embeddings 304, and processes the block input to generate the updated single embeddings 310 and the updated pair embeddings 312.

The update block 300 includes a single embedding update block 306 and a pair embedding update block 308.

The single embedding update block 306 updates the current single embeddings 302 using the current pair embeddings 304, and the pair embedding update block 308 updates the current pair embeddings 304 using the updated single embeddings 310 (i.e., that are generated by the single embedding update block 306).

Generally, the single embeddings and the pair embeddings can encode complementary information. For example, the single embeddings can encode information characterizing the features of single amino acids in the protein, and the pair embeddings can encode information about the relationships between pairs of amino acids in the protein, including the distances between pairs of amino acids in the protein structure. The single embedding update block 306 enriches the information content of the single embeddings using complementary information encoded in the pair embeddings, and the pair embedding update block 308 enriches the information content of the pair embeddings using complementary information encoded in the single embeddings. As a result of this enrichment, the updated single embeddings and the updated pair embeddings encode information that is more relevant to predicting an amino acid sequence of a protein that achieves the protein structure.

The update block 300 is described herein as first updating the current single embeddings 302 using the current pair embeddings 304, and then updating the current pair embeddings 304 using the updated single embeddings 310. The description should not be understood as limiting the update block to performing operations in this sequence, e.g., the update block could first update the current pair embeddings using the current single embeddings, and then update the current single embeddings using the updated pair embeddings.

The update block 300 is described herein as including a single embedding update block 306 (i.e., that updates the current single embeddings) and a pair embedding update block 308 (i.e., that updates the current pair embeddings). The description should not be understood to limiting the update block 300 to include only one single embedding update block or only one pair embedding update block. For example, the update block 300 can include several single embedding update blocks that update the single embeddings multiple times before the single embeddings are provided to a pair embedding update block for use in updating the current pair embeddings. As another example, the update block 300 can include several pair embedding update blocks that update the pair embeddings multiple times using the single embeddings.

The single embedding update block 306 and the pair embedding update block 308 can have any appropriate architectures that enable them to perform their described functions.

In some implementations, the single embedding update block 306, the pair embedding update block 308, or both, include one or more “self-attention” blocks. As used throughout this document, a self-attention block generally refers to a neural network block that updates a collection of embeddings, i.e., that receives a collection of embeddings and outputs updated embeddings. To update a given embedding, the self-attention block can determine a respective “attention weight”, e.g., a similarity measure, between the given embedding and each of one or more selected embeddings (e.g., the other members of the received collection of embeddings), and then update the given embedding using: (i) the attention weights, and (ii) the selected embeddings. For example an updated embedding may comprise a sum of values each derived from one of the selected embeddings and each weighted by a respective attention weight. For convenience, the self-attention block may be said to update the given embedding using attention “over” the selected embeddings.

For example, a self-attention block may receive a collection of input embeddings {xᵢ}ᵢ₌₁^{N},where N is the number of amino acids in the protein, and to update embedding xᵢ, the self-attention block may determine attention weights [a_{i,j}]ⱼ₌₁^{N} where a_{i,j} denotes the attention weight between xᵢ and xⱼ, as:

$${[ {a}_{i , j} ]}_{j = 1}^{N} = softmax ( \frac{( {W}_{q} {x}_{i} ) {K}^{T}}{c} ) \qquad ( 1 )$$

$${K}^{T} = {[ {W}_{k} {x}_{j} ]}_{j = 1}^{N} \qquad ( 2 )$$

where W_{q} and Wₖ are learned parameter matrices, softmax(·) denotes a soft-max normalization operation, and c is a constant. Using the attention weights, the self-attention layer may update embedding xᵢ as:

$${x}_{i} ← {\sum }_{j = 1 . . N} {a}_{i , j} \cdot ( {W}_{v} {x}_{j} ) \qquad ( 3 )$$

where Wᵥ is a learned parameter matrix. (W_{q}xᵢ can be referred to as the “query embedding” for input embedding xᵢ, Wₖxⱼ can be referred to as the “key embedding” for input embedding xᵢ, and Wᵥxᵢ can be referred to as the “value embedding” for input embedding xᵢ).

The parameter matrices W_{q} (the “query embedding matrix”), Wₖ (the “key embedding matrix”), and Wᵥ (the “value embedding matrix”) are trainable parameters of the self-attention block. The parameters of any self-attention blocks included in the single embedding update block 306 and the pair embedding update block 308 can be understood as being parameters of the update block 300 that can be trained as part of the end-to-end training of the protein design system described with reference to FIG. 6. Generally, the (trained) parameters of the query, key, and value embedding matrices are different for different self-attention blocks, e.g., such that a self-attention block included in the single embedding update block 306 can have different query, key, and value embedding matrices with different parameters than a self-attention block included in the pair embedding update block 308.

In some implementations, the pair embedding update block 308, the single embedding update block 306, or both, include one or more self-attention blocks that are conditioned on (dependent upon) the pair embeddings, i.e., that implement self-attention operations that are conditioned on the pair embeddings. To condition a self-attention operation on the pair embeddings, the self-attention block can process the pair embeddings to generate a respective “attention bias” corresponding to each attention weight; each attention weight may then be biased by the corresponding attention bias. For example, in addition to determining the attention weights [a_{i,j}]ⱼ₌₁^{N} in accordance with equations (1)-(2), the self-attention block can generate a corresponding set of attention biases [b_{i,j}]ⱼ₌₁^{N} where denotes the attention bias between xᵢ and xⱼ. The self-attention block can generate the attention bias b_{i,j} by applying a learned parameter matrix to the pair embedding for the pair of amino acids in the protein indexed by (i,j).

The self-attention block can determine a set of “biased attention weights” [c_{i,j}]ⱼ₌₁^{N}, where c_{i,j} denotes the biased attention weight between xᵢ and xⱼ, e.g., by summing (or otherwise combining) the attention weights and the attention biases. For example, the self-attention block can determine the biased attention weight c_{i,j} between embeddings xᵢ and xⱼ as:

c_{i,j}=a_{i,j}+b_{i,j}

where a_{i,j} is the attention weight between xᵢ and xⱼ and b_{i,j} is the attention bias between xᵢ and xⱼ. The self-attention block can update each input embedding xᵢ using the biased attention weights, e.g.:

$${x}_{i} ← {\sum }_{j = 1. . N} {c}_{i , j} \cdot ( {W}_{v} {x}_{j} ) \qquad ( 4 )$$

where Wᵥ is a learned parameter matrix.

Generally, the pair embeddings encode information characterizing the structure of the protein and the relationships between the pairs of amino acids in the structure of the protein. Applying a self-attention operation that is conditioned on the pair embeddings to a set of input embeddings allows the input embeddings to be updated in a manner that is informed by the protein structural information encoded in the pair embeddings. The update blocks of the embedding neural network can use the self-attention blocks that are conditioned on the pair embeddings to update and enrich the single embeddings and the pair embeddings themselves.

Optionally, a self-attention block can have multiple “heads” that each generate a respective updated embedding corresponding to each input embedding, i.e., such that each input embedding is associated with multiple updated embeddings. For example, each head may generate updated embeddings in accordance with different values of the parameter matrices W_{q}, Wₖ, and Wᵥ that are described with reference to equations (1)-(4). A self-attention block with multiple heads can implement a “gating” operation to combine the updated embeddings generated by the heads for an input embedding, i.e., to generate a single updated embedding corresponding to each input embedding. For example, the self-attention block can process the input embeddings using one or more neural network layers (e.g., fully connected neural network layers) to generate a respective gating value for each head. The self-attention block can then combine the updated embeddings corresponding to an input embedding in accordance with the gating values. For example, the self-attention block can generate the updated embedding for an input embedding xᵢ as:

$${\sum }_{k = 1}^{K} {α}_{k} \cdot {x}_{i}^{n e x t} \qquad ( 5 )$$

where k indexes the heads, aₖ is the gating value for head k, and xᵢ^{next} is the updated embedding generated by head k for input embedding xᵢ.

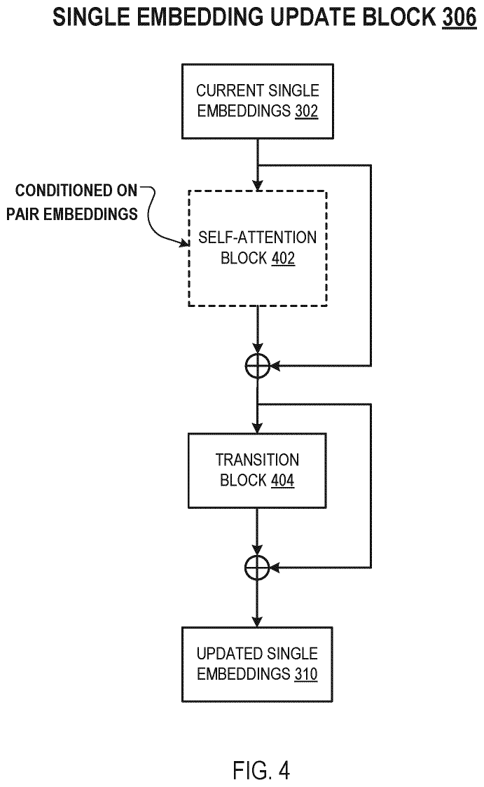

An example architecture of a single embedding update block 306 that uses self-attention blocks conditioned on the pair embeddings is described with reference to FIG. 4.

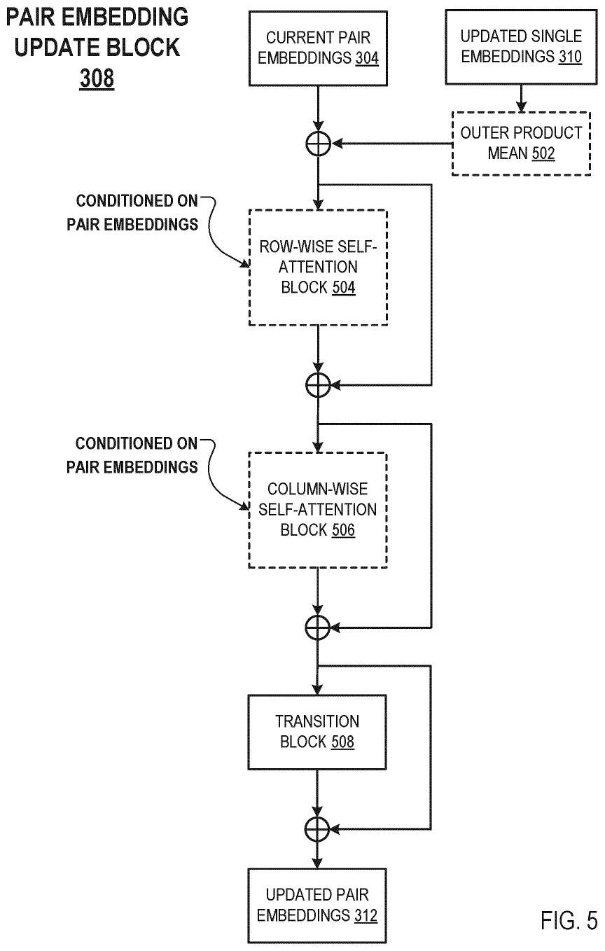

An example architecture of a pair embedding update block 308 that uses self-attention blocks conditioned on the pair embeddings is described with reference to FIG. 5. The example pair embedding update block described with reference to FIG. 5 updates the current pair embeddings based on the updated single embeddings by computing an outer product (hereinafter referred to as an “outer product mean”) of the updated single embeddings, adding the result of the outer product mean to the current pair embeddings (projected to the pair embedding dimension, if necessary), and processing the current pair embeddings using self-attention blocks.

FIG. 4 shows an example architecture of a single embedding update block 306. The single embedding update block 306 is configured to receive the current single embeddings, and to update the current single embeddings 302 based (at least in part) on the current pair embeddings.

To update the current single embeddings 302, the single embedding update block 306 updates the single embeddings using a self-attention operation that is conditioned on the current pair embeddings. More specifically, the single embedding update block 306 provides the single embeddings to a self-attention block 402 that is conditioned on the current pair embeddings, e.g., as described with reference to FIG. 3, to generate updated single embeddings. Optionally, the single embedding update block can add the input to the self-attention block 402 to the output of the self-attention block 402. Conditioning the self-attention block 402 on the current pair embeddings enables the single embedding update block 306 to enrich the current single embeddings 302 using information from the current pair embeddings.

The single embedding update block then processes the current single embeddings 302 using a transition block 404, e.g., that applies one or more fully-connected neural network layers to the current single embeddings. Optionally, the single embedding update block 306 can add the input to the transition block 404 to the output of the transition block 404. The single embedding update block can output the updated single embeddings 310 resulting from the operations performed by the self-attention block 402 and the transition block 404.

FIG. 5 shows an example architecture of a pair embedding update block 308. The pair embedding update block 308 is configured to receive the current pair embeddings 304, and to update the current pair embeddings 304 based (at least in part) on the updated single embeddings 310.

In the description which follows, the pair embeddings can be understood as being arranged into an N×N array, i.e., such that the embedding at position (i,j) in the array is the pair embedding corresponding to the amino acids at positions i and j in the amino acid sequence.

To update the current pair embeddings 304, the pair embedding update block 308 applies an outer product mean operation 502 to the updated single embeddings 310 and adds the result of the outer-product mean operation 502 to the current pair embeddings 304.

The outer product mean operation defines a sequence of operations that, when applied to the set of single embeddings, each represented as an 1×N array of embeddings, generates an N×N array of embeddings, i.e., where N is the number of amino acids in the protein. The current pair embeddings 304 can also be represented as an N×N array of pair embeddings, and adding the result of the outer product mean 502 to the current pair embeddings 304 refers to summing the two N×N arrays of embeddings.

To compute the outer product mean, the pair embedding update block 308 generates a tensor A(·), e.g., given by:

A(res1,res2,ch1,ch2)=LeftAct(res1,ch1)·RightAct(res2,ch2) (6)

where res1, res2 ∈{1, . . . , N}, ch1, ch2 ∈{1, . . . , C}, where C is the number of channels in each single embedding, LeftAct(res1, ch1) is a linear operation (e.g., a projection, e.g., defined by a matrix multiplication) applied to the channel ch1 of the single embedding indexed by “res1”, and RightAct(res2, ch2) is a linear operation (e.g., a projection, e.g., defined by a matrix multiplication) applied to the channel ch2 of the single embedding indexed by “res2”. The result of the outer product mean is generated by flattening and linearly projecting the (ch1, ch2) dimensions of the tensor A. Optionally, the pair embedding update block can perform one or more Layer Normalization operations (e.g., as described with reference to Jimmy Lei Ba et al., “Layer Normalization,” arXiv:1607.06450) as part of computing the outer product mean.

Generally, the updated single embeddings 310 encodes information about the amino acids in the amino acid sequence of the protein. The information encoded in the updated single embeddings 310 is relevant to predicting the amino acid sequence of the protein, and by incorporating the information encoded in the updated single embeddings into the current pair embeddings (i.e., by way of the outer product mean 502), the pair embedding update block 308 can enhance the information content of the current pair embeddings.

After updating the current pair embeddings 304 using the updated single embeddings (i.e., by way of the outer product mean 502), the pair embedding update block 308 updates the current pair embeddings in each row of an arrangement of the current pair embeddings into an N×N array using a self-attention operation (i.e., a “row-wise” self-attention operation) that is conditioned on the current pair embeddings. More specifically, the pair embedding update block 308 provides each row of current pair embeddings to a “row-wise” self-attention block 504 that is also conditioned on the current pair embeddings, e.g., as described with reference to FIG. 3, to generate updated pair embeddings for each row. Optionally, the pair embedding update block can add the input to the row-wise self-attention block 504 to the output of the row-wise self-attention block 504.

The pair embedding update block 308 then updates the current pair embeddings in each column of the N×N array of current pair embeddings using a self-attention operation (i.e., a “column-wise” self-attention operation) that is also conditioned on the current pair embeddings. More specifically, the pair embedding update block 308 provides each column of current pair embeddings to a “column-wise” self-attention block 506 that is also conditioned on the current pair embeddings to generate updated pair embeddings for each column. Optionally, the pair embedding update block can add the input to the column-wise self-attention block 506 to the output of the column-wise self-attention block 506.

The pair embedding update block 308 then processes the current pair embeddings using a transition block 508, e.g., that applies one or more fully-connected neural network layers to the current pair embeddings. Optionally, the pair embedding update block 308 can add the input to the transition block 508 to the output of the transition block 508. The pair embedding update block can output the updated pair embeddings 312 resulting from the operations performed by the row-wise self-attention block 504, the column-wise self-attention block 506, and the transition block 508.

FIG. 6 shows an example training system 600 for training a protein design system, e.g., the protein design system 100 described with reference to FIG. 1. The training system 600 is an example of a system implemented as computer programs on one or more computers in one or more locations in which the systems, components, and techniques described below are implemented.

The training system 600 trains the parameters of the protein design system 604. The protein design system 604 is configured to process a set of structure parameters defining a protein structure, in accordance with current values of a set of protein design system parameters, to generate data defining an amino acid sequence of a protein that is predicted to achieve the protein structure. In the description which follows, the protein design system 604 is understood to be a neural network system (i.e., a system of one or more neural networks), and the protein design system parameters include the (trainable) parameters (e.g., weights) of the protein design system 604. For example, the protein design system parameters of the protein design system described with reference to FIG. 1 include the neural network parameters of the embedding neural network 200 and of the generative neural network 106.

The training system 600 trains the protein design system 604 on a set of training examples. Each training example includes a respective set of structure parameters defining a “training” protein structure, and optionally, data defining a “target” amino acid sequence of a protein that achieves the training protein structure. The training protein structures and the corresponding target amino acid sequences can be determined through experimental techniques. Conventional physical techniques, such as x-ray crystallography, magnetic resonance techniques, or cryogenic electron microscopy (cryo-EM), may be used to measure the respective training protein structures of a plurality of proteins existing in the real world (e.g., natural proteins as defined below). Protein sequencing may be used to measure the respective target amino acid sequences of the plurality of proteins.

The training system 600 trains the protein design system 604 on the training examples using stochastic gradient descent. More specifically, at each training iteration in a sequence of training iterations, the training system 600 samples one or more training protein structures 602. The training system 600 processes the training protein structures 602 using the protein design system 604, in accordance with the current values of the protein design system parameters, to generate a respective predicted amino acid sequence 606 corresponding to each training protein structure. The training system 600 then determines gradients of an objective function that depends on the predicted amino acid sequences 606, and uses the gradients of the objective function to update the current values of the protein design system parameters. The training system 600 can determine the gradients of the objective function with respect to the protein design system parameters, e.g., using backpropagation, and can update the current values of the protein design system parameters using the update rule of any appropriate gradient descent optimization algorithm, e.g., RMSprop or Adam.

The objective function includes one or more of: (i) a sequence loss 608, (ii) a structure loss 614, and (iii) a realism loss 620, each of which will be described in more detail below. For example, the objective function may be defined as a linear combination of the sequence loss 608, the structure loss 614, and the realism loss 620, e.g., such that the objective function may be given by:

[symbol]

(PS)=a₁· [symbol] _{seq}(PS)+a₂· [symbol] _{struct}(PS)+a₃· [symbol] ᵣₑₐₗ(PS) (7)

where [symbol] (PS) denotes the objective function evaluated on predicted amino acid sequence PS, {aᵢ}ᵢ₌₁³ are scaling coefficients, [symbol] _{seq}(PS) denotes the sequence loss evaluated on predicted amino acid sequence PS, [symbol] _{struct}(PS) denotes the structure loss evaluated on predicted amino-acid sequence PS, and [symbol] ᵣₑₐₗ(PS) denotes the realism loss evaluated on predicted amino acid sequence PS.

To evaluate the sequence loss 608 for a predicted amino acid sequence 606, the training system 600 determines a similarity between: (i) the predicted amino acid sequence 606, and (ii) the corresponding target amino acid sequence for the training protein structure 602. The training system 600 may determine the similarity between a predicted amino acid sequence and a target amino acid sequence, e.g., using a cross-entropy loss. Training the protein design system 604 to minimize the sequence loss 608 encourages the protein design system 604 to generate predicted amino acid sequences that match the target amino acid sequences specified by the training examples.

To evaluate the structure loss 614 for a predicted amino acid sequence 606, the training system 600 provides the predicted amino acid sequence 606 to a protein folding neural network 610. Any protein folding neural network may be used, e.g., based on a published approach or on software such as AlphaFold2 (available open source). The protein folding neural network 610 is configured to process the predicted amino acid sequence 606 to generate structure parameters that define a predicted structure 612 of the protein having the predicted amino acid sequence 606. The training system 600 determines the structure loss 614 for the predicted amino acid sequence 606 by determining a similarity measure between: (i) the training protein structure 602, and (ii) the predicted protein structure 612.

The training system 600 can determine a similarity measure between: (i) a training protein structure 602, and (ii) a predicted protein structure 612 in any appropriate way. In one example, the training protein structure 602 can be represented by structure parameters that define the respective 3D spatial position of the alpha carbon atom in each amino acid in the training protein structure. Similarly, the predicted protein structure 612 can be represented by structure parameters that define the respective 3D spatial position of the alpha carbon atom in each amino acid in the predicted protein structure. In this example, the training system 600 can determine the similarity measure between the training protein structure and the predicted protein structure as:

$${\sum }_{a} \vert {T}_{a} - {P}_{a} \vert \qquad ( 8 )$$

where a indexes the amino acids in the protein, T a denotes the 3D spatial position of the alpha carbon atom of amino acid a as defined by the training protein structure 602, Pₐ denotes the 3D spatial position of the alpha carbon atom of amino acid a as defined by the predicted protein structure 612, and |·| denotes a distance measure, e.g., a squared Euclidean distance measure.

If the objective function includes the structure loss 614, then the training system 600 determines gradients of the structure loss 614 with respect to the protein design system parameters as part of determining gradients of the objective function. To determine gradients of the structure loss 614 with respect to the protein design system parameters, the training system 600 backpropagates the gradients of the structure loss 614 through the protein folding neural network 610 and into the neural networks of the protein design system 604. The protein folding neural network 610 itself is generally trained before being used during training of the protein design system 604, and the training system 600 does not update the parameters of the protein folding neural network 610 using gradients of the structure loss 614. That is, the training system 600 treats the parameters of the protein folding neural network 610 as static values while backpropagating gradients of the structure loss 614 through the protein folding neural network 610 into the neural networks of the protein design system 604.

The protein folding neural network 610 can have any appropriate neural network architecture that enables it to perform its described function, i.e., processing data defining an amino acid sequence of a protein to generate a set of structure parameters that define a predicted structure of the protein. For example, the protein folding neural network 610 can include any appropriate types of neural network layers (e.g., fully-connected layers, convolutional layers, or self-attention layers) connected in any appropriate configuration (e.g., as a linear sequence of layers).

Training the protein design system 604 to optimize the structure loss 614 encourages the protein design system 604 to generate predicted amino acid sequences 606 of proteins that fold into structures which match the training protein structures 602. The structure loss 614 evaluates the accuracy of the protein design system 604 in “structure space,” i.e., in the space of possible protein structures, in contrast to the sequence loss 608, which evaluates the accuracy of the protein design system 604 in “sequence space,” i.e., in the space of possible amino acid sequences. Therefore, the gradient signal generated using the structure loss 614 is complementary to the gradient signal generated using the sequence loss 608. Training the protein design system 604 using both the structure loss 614 and the sequence loss 608 can enable the protein design system 604 to achieve higher accuracy than would be achieved using structure loss 614 alone or the sequence loss 608 alone.

Generally, the structure loss 614 can be evaluated even if the target amino acid sequence for a training protein structure 602 is unknown. In contrast, the sequence loss 608 can be evaluated only if the target amino acid sequence for the training protein structure is known. Therefore, the structure loss 614 enables the protein design system 604 to be trained on a broader class of training examples than the sequence loss 608. In particular, the structure loss 614 enables the protein design system 604 to be trained on training examples that include training protein structures for which the target amino acid sequence is unknown.

The training system 600 evaluates the realism loss 620 for a predicted amino acid sequence 606 using a discriminator neural network 616. The discriminator neural network 616 is configured to process data characterizing a protein that includes: an amino acid sequence of the protein, a set of protein structure parameters defining an (actual or predicted) structure of the protein, or both, to generate a realism score for the protein. The discriminator neural network 616 is trained to generate realism scores that classify whether proteins are: (i) “synthetic” proteins, or (ii) “natural” proteins. That is, the discriminator neural network is trained to generate realism scores that define a likelihood that a protein is a synthetic protein as opposed to a natural protein.

A synthetic protein refers to a protein having an amino acid sequence that is generated by the protein design system 604.

A natural protein refers to a protein from a set of proteins that have been designated as being “realistic,” e.g., as a result of being identified as proteins that exist in the real world, such as naturally-occurring proteins that have been collected from biological systems.

To evaluate the realism loss 620 for a predicted amino acid sequence 606, the training system 600 provides the predicted amino acid sequence 606, a predicted protein structure 612 of the protein having the predicted amino acid sequence 606, or both, to the discriminator neural network 616. The training system 600 can generate the predicted protein structure 612 by processing the predicted amino acid sequence 606 using the protein folding neural network 610. The discriminator neural network 616 processes the input to generate a realism score 618 that classifies (predicts) whether the protein generated by the protein design system is a synthetic protein or a natural protein. The training system 600 determines the realism loss 620 a function of the realism score, e.g., as the negative of the realism score.

If the objective function includes the realism loss 620, then the training system 600 determines gradients of the realism loss 620 with respect to the protein design system parameters as part of determining gradients of the objective function. To determine gradients of the realism loss 620 with respect to the protein design system parameters, the training system 600 backpropagates the gradients of the realism loss 620 through the discriminator neural network 616 into the protein folding neural network 610, and through the protein folding neural network 610 into the neural networks of the protein design system 604. The training system 600 treats the parameters of the discriminator neural network 616 and the protein folding neural network 610 as static while backpropagating gradients of the realism loss 620 through them to into the neural networks of the protein design system 604.

The training system 600 trains the discriminator neural network 616 to perform the classification task of discriminating between synthetic proteins and natural proteins. For example, the training system 600 can train the discriminator neural network 616 to generate a first value (e.g., the value 0) by processing data characterizing a synthetic protein, and to generate a second value (e.g., the value 1) by processing data characterizing a natural protein. The training system 600 can generate data characterizing a synthetic protein by processing a training protein structure 602 using the protein design system 604 to generate a predicted amino acid sequence 606 of the synthetic protein, and optionally, processing the predicted amino acid sequence 606 using the protein folding neural network 610 to generate a predicted protein structure of the synthetic protein. The training system 600 can train the discriminator neural network 616 using any appropriate training technique, e.g., stochastic gradient descent, to optimize any appropriate objective function, e.g., a binary cross-entropy objective function.

As the protein design system 604 is trained, the values of the protein design system parameters are iteratively adjusted, thereby altering the characteristics of the synthetic proteins being generated by the protein design system 604. To enable the discriminator neural network 616 to adapt to the changing characteristics of the synthetic proteins being generated by the protein design system 604, the training system 600 can train the discriminator neural network 616 concurrently with the protein design system 604. For example, the training system 600 can alternate between training the protein design system 604 and the discriminator neural network 616. Each time the training system 600 is tasked with training the discriminator neural network 616, the training system 600 can generate new synthetic proteins in accordance with the most recent values of the protein design system parameters, and train the discriminator neural network on the new synthetic proteins.

The discriminator neural network 616 can have any appropriate neural network architecture that enables it to perform its described function, i.e., processing data characterizing a protein to generate a realism score. In particular, the discriminator neural network can include any appropriate neural network layers, e.g., convolutional layers, fully-connected layers, self-attention layers, etc., connected in any appropriate configuration (e.g., as a linear sequence of layers).

In some implementations, the discriminator neural network 616 is configured to process data characterizing protein fragments with a predefined length, e.g., of 5 amino acids, 10 amino acids, or 15 amino acids. To generate a realism score for a protein with a length that exceeds the predefined length that the discriminator neural network is configured to receive, the training system 600 can partition the amino acid sequence of the protein into multiple sub-sequences having the predefined length. The training system 600 can process data characterizing each amino acid sub-sequence (e.g., the amino acids in the sub-sequence and the structure parameters defining the structure of the sub-sequence) using the discriminator neural network to generate a respective realism score. The training system 600 can then combine (e.g., average) the realism scores for the amino acid sub-sequences to generate a realism score for the original protein.

Training the protein design system 604 to optimize the realism score 618 can improve the performance (e.g., accuracy) of the protein design system 604 by encouraging the protein design system 604 to generate proteins having the characteristics of the real proteins that exist in the real world. In particular, the discriminator neural network 616 can learn to implicitly recognize complex, high-level features of realistic proteins, and the protein design system 604 can learn to generate proteins that share these features.

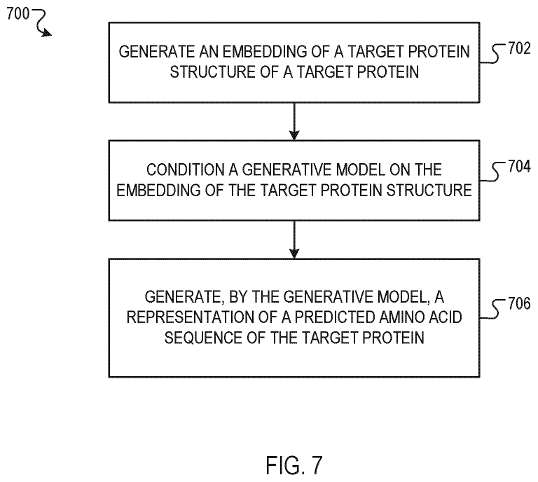

FIG. 7 is a flow diagram of an example process 700 for determining a predicted amino acid sequence of a target protein having a target protein structure. For convenience, the process 700 will be described as being performed by a system of one or more computers located in one or more locations. For example, a protein design system, e.g., the protein design system 100 of FIG. 1, appropriately programmed in accordance with this specification, can perform the process 700.

The system processes an input characterizing the target protein structure of the target protein using an embedding neural network to generate an embedding of the target protein structure of the target protein (702).

The system conditions a generative neural network on the embedding of the target protein structure (704).

The system generates, by the generative neural network conditioned on the embedding of the target protein structure, a representation of the predicted amino acid sequence of the target protein (706).

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method performed by one or more data processing apparatus, the method comprising:
processing an input characterizing a target protein structure of a target protein using an embedding neural network having a plurality of embedding neural network parameters to generate an embedding of the target protein structure of the target protein;
determining a predicted amino acid sequence of the target protein based on the embedding of the target protein structure, comprising:
conditioning a generative neural network having a plurality of generative neural network parameters on the embedding of the target protein structure; and
generating, by the generative neural network conditioned on the embedding of the target protein structure, a representation of the predicted amino acid sequence of the target protein;
processing the representation of the predicted amino acid sequence using a protein folding neural network to generate a representation of a predicted protein structure of a protein having the predicted amino acid sequence;
determining a structural similarity measure between: (i) the predicted protein structure of the protein having the predicted amino acid sequence, and (ii) the target protein structure;
determining gradients of the structural similarity measure with respect to the embedding neural network parameters and the generative neural network parameters; and
adjusting current values of the embedding neural network parameters and the generative neural network parameters using the gradients of the structural similarity measure.

2. The method of claim 1, wherein determining gradients of the structural similarity measure with respect to the embedding neural network parameters and the generative neural network parameters comprises:
backpropagating gradients of the structural similarity measure through the protein folding neural network into the generative neural network and the embedding neural network.

3. The method of claim 1, further comprising:
processing the representation of the predicted protein structure of the protein having the predicted amino acid sequence using a discriminator neural network to generate a realism score that defines a likelihood that the predicted amino acid sequence was generated using the generative neural network;
determining gradients of the realism score with respect to the embedding neural network parameters and the generative neural network parameters; and
adjusting current values of the embedding neural network parameters and the generative neural network parameters using the gradients of the realism score.

4. The method of claim 3, wherein determining gradients of the realism score with respect to the embedding neural network parameters and the generative neural network parameters comprises:
backpropagating gradients of the realism score through the discriminator neural network and the protein folding neural network into the generative neural network and the embedding neural network.

5. The method of claim 3, wherein generating the realism score comprises processing an input that includes both: (i) the representation of the predicted protein structure having the predicted amino acid sequence, and (ii) the representation of the predicted amino acid sequence, using the discriminator neural network.

6. The method of claim 1, further comprising:
determining a sequence similarity measure between: (i) the predicted amino acid sequence of the target protein, and (ii) a target amino acid sequence of the target protein;
determining gradients of the sequence similarity measure with respect to the embedding neural network parameters and the generative neural network parameters; and
adjusting current values of the embedding neural network parameters and the generative neural network parameters using the gradients of the sequence similarity measure.

7. The method of claim 1, wherein the embedding neural network input characterizing the target protein structure comprises: (i) a respective initial pair embedding corresponding to each pair of amino acids in the target protein that characterizes a distance between the pair of amino acids in the target protein structure, and (ii) a respective initial single embedding corresponding to each amino acid in the target protein.

8. The method of claim 7, wherein the embedding neural network comprises a sequence of update blocks,
wherein each update block has a respective set of update block parameters and performs operations comprising:
receiving current pair embeddings and current single embeddings;
updating the current single embeddings, in accordance with values of the update block parameters of the update block, based on the current pair embeddings; and
updating the current pair embeddings, in accordance with the values of the update block parameters of the update block, based on the updated single embeddings;
wherein a first update block in the sequence of update blocks receives the initial pair embeddings and the initial single embeddings; and
wherein a final update block in the sequence of update blocks generates final pair embeddings and final single embeddings.

9. The method of claim 8, wherein generating the embedding of the target protein structure of the target protein comprises:
generating the embedding of the target protein structure of the target protein based on the final pair embeddings, the final single embeddings, or both.

10. The method of claim 8, wherein updating the current single embeddings based on the current pair embeddings comprises:
updating the current single embeddings using attention over the current single embeddings, wherein the attention is conditioned on the current pair embeddings.

11. The method of claim 10, wherein updating the current single embeddings using attention over the current single embeddings comprises:
generating, based on the current single embeddings, a plurality of attention weights;
generating, based on the current pair embeddings, a respective attention bias corresponding to each of the attention weights;
generating a plurality of biased attention weights based on the attention weights and the attention biases; and
updating the current single embeddings using attention of the current single embeddings based on the biased attention weights.

12. The method of claim 8, wherein updating the current pair embeddings based on the updated single embeddings comprises:
applying a transformation operation to the updated single embeddings; and
updating the current pair embeddings by adding a result of the transformation operation to the current pair embeddings.

13. The method of claim 12, wherein the transformation operation comprises an outer product operation.

14. The method of claim 12, wherein updating the current pair embeddings based on the updated single embeddings further comprises, after adding the result of the transformation operation to the current pair embeddings:
updating the current pair embeddings using attention over the current pair embeddings, wherein the attention is conditioned on the current pair embeddings.

15. The method of claim 1, wherein generating, by the generative neural network conditioned on the embedding of the target protein structure, the representation of the predicted amino acid sequence of the target protein comprises:
processing the embedding of the target protein structure to generate data defining parameters of a probability distribution over a latent space;
sampling a latent variable from the latent space in accordance with the probability distribution over the latent space; and
processing the latent variable sampled from the latent space to generate the representation of the predicted amino acid sequence.

16. The method of claim 1, wherein generating, by the generative neural network conditioned on the embedding of the target protein structure, the representation of the predicted amino acid sequence of the target protein comprises, for each position in the predicted amino acid sequence:
processing: (i) the embedding of the target protein structure, and (ii) data defining amino acids at any preceding positions in the predicted amino acid sequence, to generate a probability distribution over a set of possible amino acids; and
sampling an amino acid for the position in the predicted amino acid sequence from the set of possible amino acids in accordance with the probability distribution over the set of possible amino acids.

17. The method of claim 1, further comprising obtaining a representation of a three-dimensional shape and size of a surface portion of a target body, and obtaining the target protein structure as a structure including a portion which has a shape and size complementary to the shape and size of the surface portion of the target body.

18-24. (canceled)

25. A system comprising:
one or more computers; and
one or more storage devices communicatively coupled to the one or more computers, wherein the one or more storage devices store instructions that, when executed by the one or more computers, cause the one or more computers to perform operations comprising:
processing an input characterizing a target protein structure of a target protein using an embedding neural network having a plurality of embedding neural network parameters to generate an embedding of the target protein structure of the target protein;
determining a predicted amino acid sequence of the target protein based on the embedding of the target protein structure, comprising:
conditioning a generative neural network having a plurality of generative neural network parameters on the embedding of the target protein structure; and
generating, by the generative neural network conditioned on the embedding of the target protein structure, a representation of the predicted amino acid sequence of the target protein;
processing the representation of the predicted amino acid sequence using a protein folding neural network to generate a representation of a predicted protein structure of a protein having the predicted amino acid sequence;
determining a structural similarity measure between: (i) the predicted protein structure of the protein having the predicted amino acid sequence, and (ii) the target protein structure;
determining gradients of the structural similarity measure with respect to the embedding neural network parameters and the generative neural network parameters; and
adjusting current values of the embedding neural network parameters and the generative neural network parameters using the gradients of the structural similarity measure.

26. One or more non-transitory computer storage media storing instructions that when executed by one or more computers cause the one or more computers to perform operations comprising:
processing an input characterizing a target protein structure of a target protein using an embedding neural network having a plurality of embedding neural network parameters to generate an embedding of the target protein structure of the target protein;
determining a predicted amino acid sequence of the target protein based on the embedding of the target protein structure, comprising:
conditioning a generative neural network having a plurality of generative neural network parameters on the embedding of the target protein structure; and
generating, by the generative neural network conditioned on the embedding of the target protein structure, a representation of the predicted amino acid sequence of the target protein;
processing the representation of the predicted amino acid sequence using a protein folding neural network to generate a representation of a predicted protein structure of a protein having the predicted amino acid sequence;
determining a structural similarity measure between: (i) the predicted protein structure of the protein having the predicted amino acid sequence, and (ii) the target protein structure;
determining gradients of the structural similarity measure with respect to the embedding neural network parameters and the generative neural network parameters; and
adjusting current values of the embedding neural network parameters and the generative neural network parameters using the gradients of the structural similarity measure.

27. The non-transitory computer storage media of claim 26, wherein determining gradients of the structural similarity measure with respect to the embedding neural network parameters and the generative neural network parameters comprises:
backpropagating gradients of the structural similarity measure through the protein folding neural network into the generative neural network and the embedding neural network.
